(19) United States
(12) Patent Application Publication
(10) Pub. No.: US 2023/0309842 A1
(43) Pub. Date: Oct. 5, 2023

(54) OCCLUSION BALLOONS AND DISTAL THROMBECTOMY CATHETERS WITH BLOOD FLOW SENSORS AND AUTOMATED INFLATION

(71) Applicant: University of Utah Research Foundation, Salt Lake City, UT (US)

(72) Inventors: Matthew ALEXANDER, Salt Lake City, UT (US); Ramesh GRANDHI, Salt Lake City, UT (US); Austin JOHNSON, Salt Lake City, UT (US)

(21) Appl. No.: 18/022,411

(22) PCT Filed: Aug. 21, 2021
(86) PCT No.: PCT/US2021/047036

§ 371 Date: Feb. 21, 2023

Related U.S. Application Data
(60) Provisional application No. 63/068,771, filed on Aug. 21, 2020.

(51) Int. Cl. A61B 5/021; A61B 5/0215; A61B 17/12

(52) U.S. Cl. CPC A61B 5/02141; A61B 5/0215; A61B 17/12045; A61B 5/02233

(57) ABSTRACT

Catheter devices and methods are disclosed and described. A catheter device (100) can include a longitudinal lumen (104) and having a proximal end (104a) and a distal end (104b). The distal end (104b) is capable of insertion into at least the internal carotid artery. The catheter device (100) can include an occlusion balloon (120b) connected to the distal end (104b) and operable to occlude blood flow in a blood vessel (102) by inflation and deflation using a pressurization fluid. The catheter device (100) can include a pressure sensor (110b) associated with the distal end (104b) and operable to measure blood pressure data from at least one of a downstream (103b) and upstream (103a) location of the occlusion balloon (120b) and transmit the blood pressure data to a controller (150). The occlusion balloon (120b) can be operable to inflate or deflate based on inflation control information. The catheter device (100) can be used to treat cerebral thrombectomy in a subject.

### RELATED APPLICATION

This application claims the benefit of U.S. Provisional Patent Application Ser. No. 63/068,771, filed on Aug. 21, 2020, which is incorporated herein by reference.

### BACKGROUND

In the United States, someone dies of a stroke every 4 minutes. Stroke is the fifth leading cause of death for Americans, representing 1 out of every 20 deaths, and is one of the leading causes of long-term disability. Each year about 800,000 people in the United States have a stroke—87% of which are classified as acute ischemic strokes (AIS). When recognized early, long-term brain damage from these ischemic strokes can be moderated using therapies aimed at removing the clot and restoring blood flow termed “mechanical thrombectomy.”

Nonetheless, ineffective clot retrieval, clot fragmentation, and embolization during mechanical thrombectomy for large vessel occlusions can result in life-threatening conditions. Multiple devices and techniques have been used for mechanical thrombectomy, but not all mechanical thrombectomy procedures are successful. In some cases, the clot cannot be safely removed with these technologies. In other cases, the clot fragments, and pieces migrate downstream into a vessel from which it is not safe to remove them, frequently leading to irreversible tissue damage and stroke.

### SUMMARY

In one embodiment, a catheter device can comprise a catheter including a longitudinal lumen and having a proximal end and a distal end. In one aspect, the distal end is capable of insertion into at least the internal carotid artery. In another aspect, the catheter device can include an occlusion balloon connected to the distal end and operable to occlude blood flow in a blood vessel by inflation and deflation using a pressurization fluid. In another aspect, the catheter device can include a pressure sensor associated with the distal end and operable to measure blood pressure data from at least one of a downstream and upstream location of the occlusion balloon and transmit the blood pressure data to a controller. In yet another aspect, the controller can be operable to receive the blood pressure data from the pressure sensor, generate inflation control information based on the blood pressure data, and transmit the inflation control information to the occlusion balloon. In one aspect, the occlusion balloon can be operable to inflate or deflate based on the inflation control information by changing a selected volume of the pressurization fluid within the occlusion balloon.

In another embodiment, a method of using a catheter device to treat cerebral thrombectomy in a subject can comprise inserting a catheter device into a cerebral vasculature of a subject such that a distal end of the catheter device is adjacent a patient thrombus. In one aspect, the method can comprise measuring blood pressure data using a pressure sensor located at least at one of a downstream and upstream position of an occlusion balloon of the catheter device. In another aspect, the method can comprise inflating or deflating the occlusion balloon based on the blood pressure data to achieve a desired degree of occlusion.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1 illustrates a catheter device including an occlusion balloon and a pair of pressure sensors positioned in a blood vessel near a blood clot in accordance with an example.

FIG. 2 illustrates a subject connected to a catheter device in accordance with an example.

FIG. 3a μlustrates a catheter device including a pair of occlusion balloons and a pair of pressure sensors positioned in a blood vessel near a blood clot in accordance with an example.

FIG. 3b μlustrates a catheter device including a pair of occlusion balloons, a pair of pressure sensors, and a stent in which the catheter device is positioned in a blood vessel near a blood clot in accordance with an example.

FIG. 3c μlustrates a catheter device including a pair of occlusion balloons, a pair of pressure sensors, and a suction source in which the catheter device is positioned in a blood vessel near a blood clot in accordance with an example.

FIG. 3d μlustrates a catheter device including an occlusion balloon, a pair of pressure sensors, and a stent in which the catheter device is positioned in a blood vessel near a blood clot in accordance with an example.

FIG. 3e μlustrates an outer catheter containing a catheter device including a pair of occlusion balloons and a pair of pressure sensors positioned in a blood vessel near a blood clot in accordance with an example.

FIG. 4 illustrates functionality for controlling occlusion balloon inflation in accordance with an example.

FIG. 5 illustrates a general computing system or device 800 that can be employed in accordance with an example.

FIG. 6 illustrates a wireless device, such as a user equipment (UE), a mobile station (MS), a mobile wireless device, a mobile communication device, a tablet, a handset, or other type of wireless device in accordance with an example.

FIG. 7 depicts a flowchart for using a catheter device in accordance with an example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an edge” includes reference to one or more of such surfaces and reference to “the sensor” refers to one or more of such features.

As used herein with respect to an identified property or circumstance, “substantially” refers to a degree of deviation that is sufficiently small so as to not measurably detract from the identified property or circumstance. The exact degree of deviation allowable may in some cases depend on the specific context. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, the terms “treat,” “treatment,” or “treating” and the like refers to administration of a therapeutic agent or therapeutic action to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, a “subject” refers to an animal. In one aspect the animal may be a mammal. In another aspect, the mammal may be a human.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, a stroke, an asthma attack, bronchitis, a heart attack, pneumonia, and the like. Some examples of chronic conditions can include without limitation, arthritis, diabetes, hypertension, high cholesterol, and the like.

As used herein, comparative terms such as “increasing,” “increased,” “decreasing,” “decreased,” “better,” “worse,” “higher,” “lower,” “enhancing,” “enhanced,” “maximizing,” “maximized,” “minimizing,” “minimized,” and the like refer to a property, result, or effect of a device, composition, formula, component, treatment, regimen, method, or activity that is measurably different from a property, result, or effect of other devices, compositions, formulas, components, treatments, regimens, methods, or activities. Furthermore, comparative terms can refer to a different biological state, presence, absence, activity level, or operation that is measurably different than an endogenous biological state, presence, absence, activity level, or operation. Comparative terms can be used to indicate differences in a surrounding or adjacent area, for example, regions of tissue. Comparative terms can also be used to indicate differences in chemical or biological structure or activity (e.g., therapeutic activity or effectiveness). Additionally, comparative terms can be used to indicate differences in biologic or physiologic result, activity, or status as compared to a previous, or other biologic or physiologic result, activity, or status. In some cases, comparison may be made simply between the points in time (e.g., endogenous state versus treated state). In other cases, the comparison can be made between the results achieved by two different applied formulations or treatment.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein.

The term “coupled,” as used herein, is defined as directly or indirectly connected in a biological, chemical, mechanical, electrical, or nonelectrical manner. “Directly coupled” structures or elements are in contact with one another and are attached. “Fluidly coupled” objects, structures, or components are in a sufficient relationship so as to allow movement or transfer of fluid from one of the objects, structures, or components to the other. Objects described herein as being “adjacent to” each other may be in physical contact with each other, in close proximity to each other, or in the same general region or area as each other, as appropriate for the context in which the phrase is used.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Over the last two decades, the mechanical thrombectomy device market has witnessed a surge of technological innovations that have shaped product design. In the late 1990's, endovascular treatment for acute stroke focused on trying to break apart the clot within the vessel using aspiration, chemical compounds, or both, but this approach can result in clots that become increasingly difficult to remove when they are fragmented, thereby leading to poor clinical outcomes. In some cases, these small fragments travel further into the brain and introduce additional complications.

Treatment with thrombectomy to treat large vessel occlusion can utilize a balloon guide catheter (BGC) in a cervicocerebral artery. Most times, the balloon guide catheter can be advanced through an introducer sheath in the common femoral artery, retrograde through the aorta, and into the common carotid to the internal carotid artery (ICA). The balloon can then be inflated in the ICA, which momentarily stops blood flow while the thrombectomy is performed. The use of a balloon guide catheter can increase first pass success rates, increase total clot removal, prevent migration of the clot to new vessels, and result in better long term neurological outcomes.

Despite data supporting the use of balloon guide catheters that arrest flow in the vessel while the clot is removed, these devices inflate a balloon in the mid-neck while the clot is removed at the base of the brain. Even though flow arrest can increase success and lead to better patient morbidity and mortality outcomes, there are a fair number of cases in which the treatment is not successful.

There are several limitations to these BGCs that limit their usefulness. The small size of the balloon requires the use of a high concentration of contrast in the inflation medium to aid in visualization which can be difficult to inflate and deflate with, at least partially due to relatively high viscosity of such contrast. In other words, inflation and deflation times can be long, and deflation can be difficult which can prolong the total duration of the procedure. The overall size (e.g., width) and stiffness of the conventional BGC allows advancement into the ICA but no further. More distal advancement that is closer to the point of occlusion would be beneficial but is not possible with these BGCs. Obtaining occlusion with a balloon more distally is complicated by attempting to inflate a balloon in the M1 segment of the middle cerebral artery (MCA), which can have catastrophic consequences if not performed delicately to prevent vessel injury. Access through the radial artery instead of the femoral artery could result in a reduced risk of injury, but BGCs are too large for use in the radial artery unless they are used without an introducer sheath—an omission that can lead to complications.

In one embodiment, a catheter device can comprise a catheter including a longitudinal lumen and having a proximal end and a distal end. In one aspect, the distal end is capable of insertion into at least the internal carotid artery. In another aspect, the distal end is sized and designed to allow insertion into vessels smaller than the ICA. In another aspect, the catheter device can include an occlusion balloon connected to the distal end and operable to occlude blood flow in a blood vessel by inflation and deflation using a pressurization fluid.

In another aspect, the catheter device can include a pressure sensor associated with the distal end and operable to measure blood pressure data from at least one of a downstream and upstream location of the occlusion balloon and transmit the blood pressure data to a controller. In yet another aspect, the controller can be operable to receive the blood pressure data from the pressure sensor, generate inflation control information based on the blood pressure data, and transmit the inflation control information to the occlusion balloon. In one aspect, the occlusion balloon can be operable to inflate or deflate based on the inflation control information by changing a selected volume of the pressurization fluid within the occlusion balloon.

In another embodiment, a method of using a catheter device to treat cerebral thrombectomy in a subject can comprise inserting a catheter device into a cerebral vasculature of a subject such that a distal end of the catheter device is adjacent a patient thrombus. In one aspect, the method can comprise measuring blood pressure data using a pressure sensor located at least at one of a downstream and upstream position of an occlusion balloon of the catheter device. In another aspect, the method can comprise inflating or deflating the occlusion balloon based on the blood pressure data to achieve a desired degree of occlusion.

The catheter device disclosed herein can include several components (e.g., an intermediate aspiration catheter, a retrievable stent, and occlusive balloons). One balloon can inflate around the distal end of the aspiration catheter to occlude flow where the thrombectomy is performed. At an end of the retrievable stent, another balloon can inflate on the inside of the stent. These two balloons can remain inflated as the system is pulled from the patient's body to perform the thrombectomy. The clot being removed can be trapped between the balloons.

To provide safety, blood flow and pressure sensors can be embedded in the devices (both the catheter and retrievable stent) to sense when blood flow halts due to balloon occlusion. The balloon can be inflated by a closed system that stops inflation when a safe and effective amount of inflation is achieved. This system can prevent unsafe overinflation of the balloons to prevent damage to the vessel. Additionally, this can be a dynamic device that maintains optimum inflation throughout the pull so that the balloon volumes change to match the change in vessel caliber as the devices are removed from the patient. Similarly, the device can dynamically and actively maintain safe inflation during use which can include shifting of the device relative to vessel walls. For example, during use the device may shift upstream or downstream slightly where vessel elasticity and/or vessel inner diameter increases or decreases. The device can provide for active and dynamic adjustment of inflation to compensate for a loss of flow blockage or an undesirable increase in pressure against vessel walls.

These properties (sensing of blood flow and pressure, and dynamic inflation of one or more balloons) can provide flow arrest at the site of the clot and allow the clot to be trapped between balloons in addition to the standard approach of retrieving the clot with a combination of a retrievable stent and aspiration. This approach can provide additional benefits in the intracranial circulation because of the real-time feedback used to maintain balloon inflation to a safe and occlusive degree. With this general invention background, the following discussion presents specific examples which further illustrate these concepts.

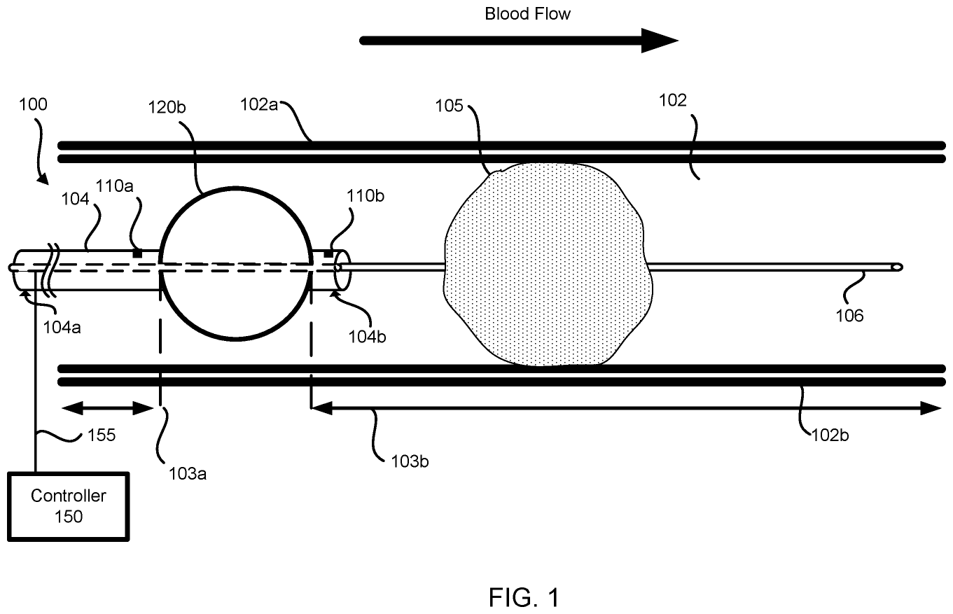

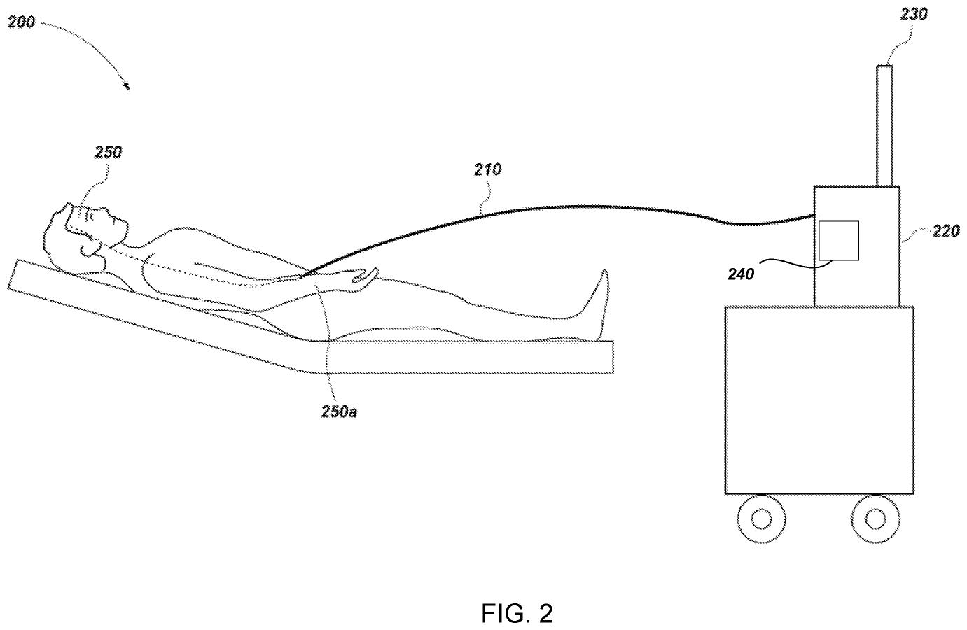

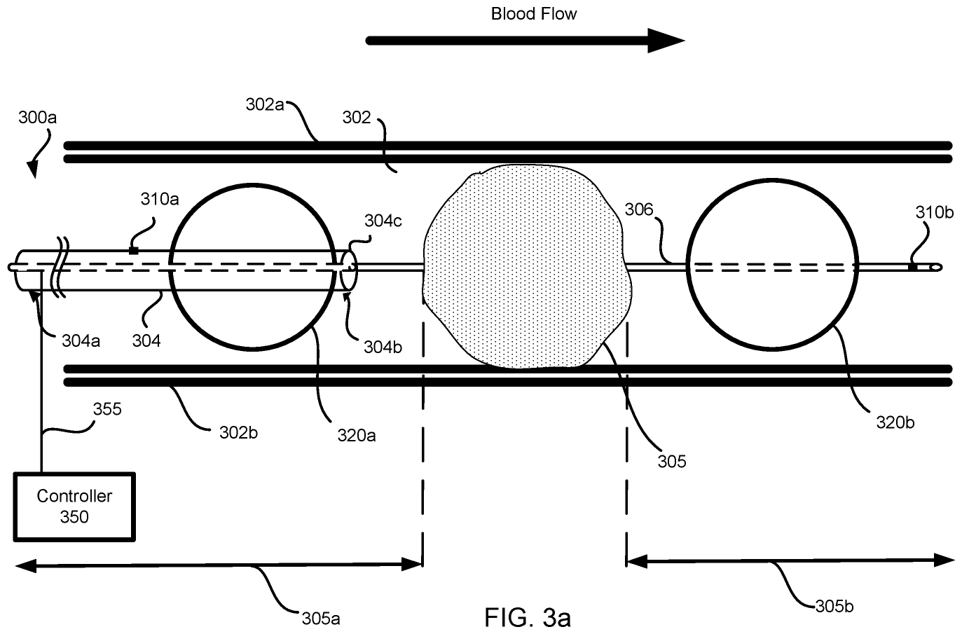

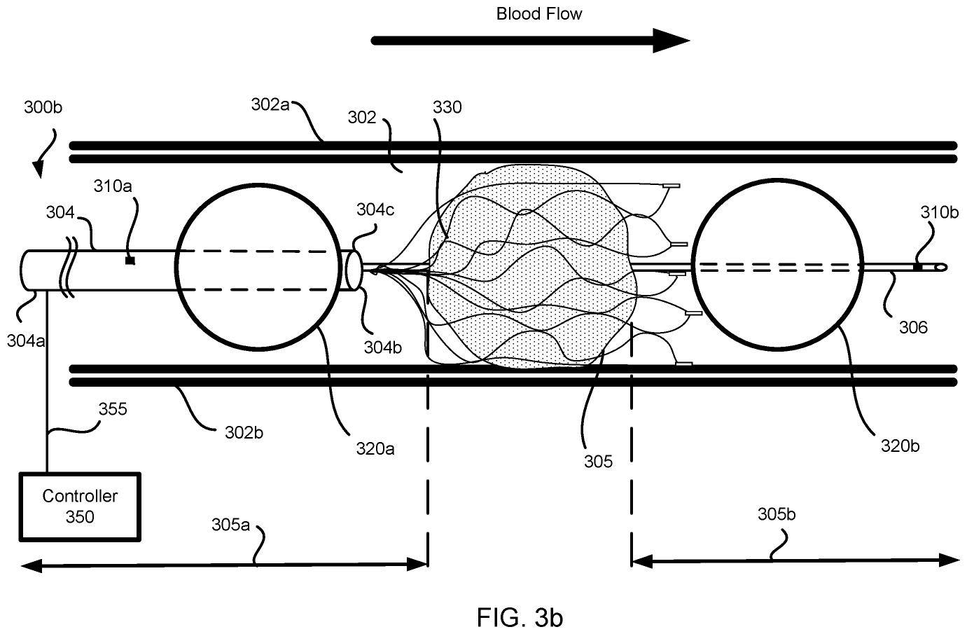

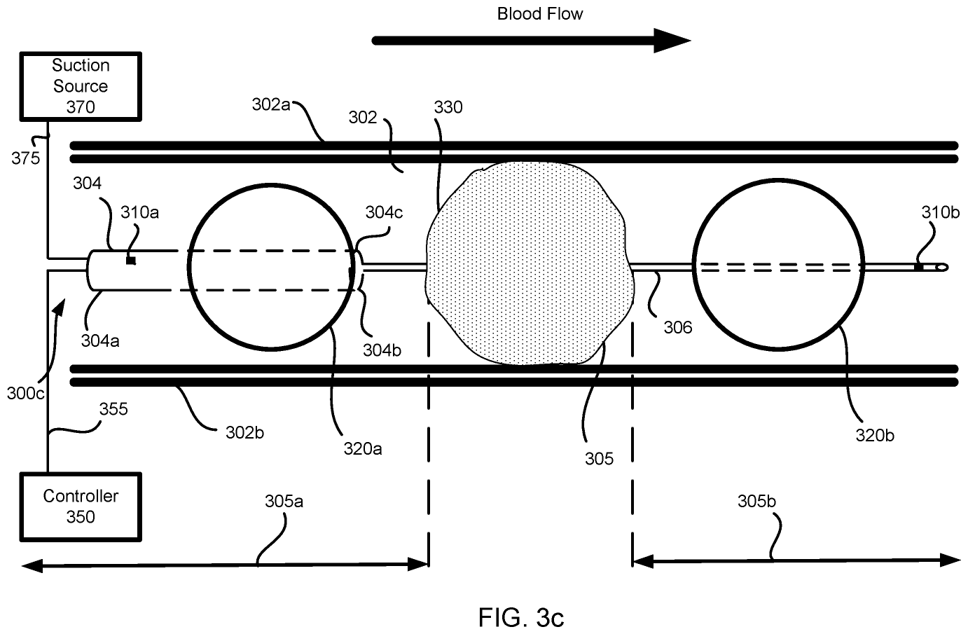

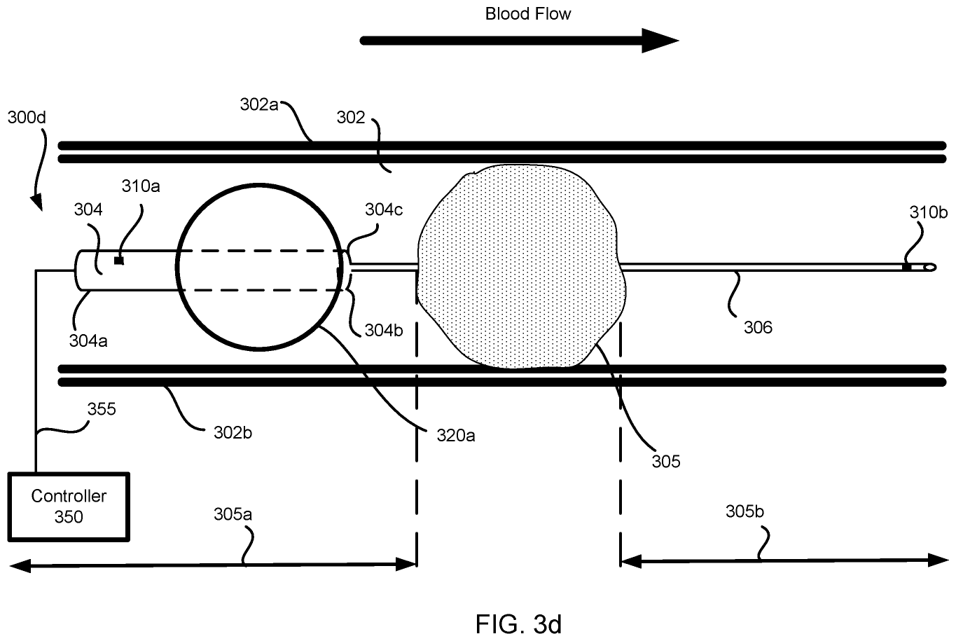

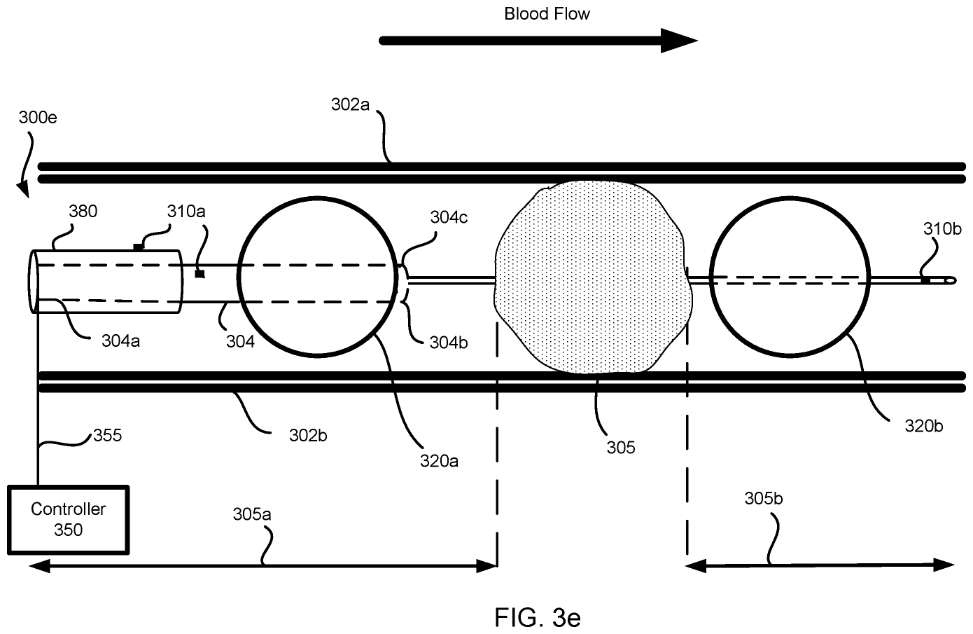

In one embodiment, as illustrated in FIG. 1, the catheter device 100 can include an occlusion balloon 120b, one or more pressure sensors 110a, 110b, and a controller 150 electrically coupled 155 to the catheter 101. In one aspect, the catheter device 100 can include a longitudinal lumen 104 having a proximal end 104a and a distal end 104b. Note that the proximal end 104a μlustrated is truncated and extends from the target location to the insertion point (see FIG. 2). An optional catheter 106 can be inserted along the vessel pathway separately or contemporaneously with the longitudinal lumen 104. The guide wire 106 can further optionally include an inflation lumen to facilitate inflation of a second occlusion balloon (as more fully described in FIG. 3A). In one aspect, the distal end 104b can be capable of insertion into at least the internal carotid artery, and in some cases into vessels smaller than the ICA. As a general guideline, an outer diameter of the longitudinal lumen 104 can be from 1 mm to 3 mm, often from 1 mm to 2 mm, and in some cases 1.8 mm to 1.9 mm. Further, the guide wire can generally have an outer diameter of 0.5 to 2 mm, and in some cases 0.5 mm to 1.6 mm. Similarly, the catheter and lumen can be formed of a material and of dimensions to allow sufficient flexibility to avoid damage to ICA and smaller vessels.

In another aspect, the occlusion balloon 120b can be connected to the distal end 104b of the guide wire and operable to occlude blood flow in a blood vessel 102 (contained by vessel walls 102a and 102b) by inflation and deflation using a pressurization fluid. The occlusion balloon can be connected to the lumen as an integrated part of the lumen, or attached to an outer surface of the lumen. In one example, the occlusion balloon can be formed from a resilient polymer bladder. For example, the resilient bladder can be glued, crimped or otherwise secured to the outer surface. Corresponding pressurization fluid holes can be located on the lumen and within the bladder to allow pressurization fluid to be introduced or withdrawn from the bladder.

In another aspect, the pressure sensor 110b can be associated with the distal end 104b and operable to measure blood pressure data from at least one of a downstream 103b and upstream 103a location of the occlusion balloon 120b and transmit the blood pressure data to a controller 150. Pressure sensors can be fully or at least partially embedded in a wall of the lumen, or attached to an outer surface of the lumen.

In yet another aspect, the controller 150 can be operable to receive the blood pressure data from the one or more pressure sensors 110a, 110b, generate inflation control information based on the blood pressure data, transmit the inflation control information to a pressurization fluid source where a pump, valve or other flow control mechanism can be used to introduce additional fluid to the occlusion balloon 120b or to remove fluid from the occlusion balloon 120b. In one more aspect, the occlusion balloon 120b can be operable to inflate or deflate based on the inflation control information by changing a selected volume of the pressurization fluid within the occlusion balloon 120b. Any suitable pressurization fluid can be used such as, but not limited to, saline solution, CO₂, and the like. In one example, the fluid can be a saline solution.

In another embodiment, a method of using the catheter device 100 to treat cerebral thrombectomy in a subject can comprise inserting a catheter device 100 into a cerebral vasculature of a subject such that a distal end 104b of the catheter device 100 is adjacent a patient thrombus 105. In one aspect, the method can comprise measuring blood pressure data using one or more pressure sensors 110a, 110b located at least at one of a downstream 103b and upstream position 103a of an occlusion balloon 120a of the catheter device 100. In another aspect, the method can comprise inflating or deflating the occlusion balloon 120a based on the blood pressure data to achieve a desired degree of occlusion.

As one example method, a guide catheter having an optional balloon on the tip can be oriented in an extracranial location and is intended to remain stationary. The guide catheter balloon can be inflated and a catheter device as described herein can be advanced to the clot. Alternatively, the guide catheter balloon can be omitted in favor of the occlusion balloon of the catheter device 100. Optionally, a second microcatheter can also be used to pass the clot and then deploy a stentriever. The distal access with a balloon on the catheter device 100 and the dynamic inflation can safely maintain flow arrest throughout the procedure of withdrawing the catheters to remove the clot.

In another alternative, the method can include inflation of a balloon on a microcatheter distal to the thrombus. For example, FIG. 3a μlustrates such an alternative as occlusion balloon 320b. The balloon can then be inflated dynamically to maintain proper inflation to withdraw and pull the clot together with the balloon until reaching a larger catheter.

### Catheter Device

Many balloon-guide catheters lack a physical mechanism to prevent fragments of clots from moving deeper into the vasculature during a cerebral thrombectomy procedure. These clot fragments can block small vessels in the brain leading to further brain injury. The use of a distal balloon can enhance clot capture by preventing fragments from passing the balloon. Further, the placement of one or more pressure sensors at the one or more balloons (e.g., proximal and distal balloons) at the site of the clot can provide real-time blood flow analysis, data on balloon vessel occlusion, and data on vascular stability. In conjunction with the pressure sensor feedback system, the one or more balloons can modulate inflation-deflation settings according to the blood pressure-flow profile. This can provide a consistent seal as a clinician retracts the clot from small cerebral arteries to vessels of larger diameter (e.g., common carotid artery). For example, vessels having an inner diameter less than about 6 mm and in some cases less than 4 mm can be reached without damage. Advantageously, the catheter device can reach vessels having an inner diameter of less than 5 mm, in some cases less than 4 mm, other cases 3 mm, and most often not less than 2 mm.

In one embodiment, as generally illustrated in FIG. 2, a catheter device system 200 can include a catheter device 210 that can provide dynamic automated changes in balloon inflation based on feedback that is generated from a controller 220 through adjustment of pressurization fluid volumes from a fluid source 240. The balloon can be positioned using either radial 250a or femoral arterial access in a subject 250. In addition to housing the balloon, the catheter device 210 can also allow for access of the cervicocerebal arteries for performance of angiography and mechanical thrombectomy. For radial access 250a, the catheter device 210 can also measure markers of perfusion to prevent ischemia to the upper extremity. For example, a waveform of the vessel pressure tracing can be analyzed. A tardus parvus waveform on the downstream transducer would indicate upstream occlusion. This can be compared with an upstream sensor in the brachial artery. If there is increased resistive index or waveform findings of “knocking” or “stump-thump,” occlusion of the supply to the upper extremity can be suspected. Absence of such findings would provide increased confidence that there is preserved flow via collaterals in the ulnar artery. The catheter device 210 can remain in place following removal of endovascular hardware used for angiography and mechanical thrombectomy. Regardless, radial pressure sensors can be oriented to be adjacent the radial artery and the brachial arteries. As a general guideline, such sensors can be oriented about 20 cm and 40 cm from a hub (e.g. bifurcated luer hub) at a proximal end of the catheter device. A hydrophilic coating can be provided along an entire length of an exterior surface of the longitudinal lumen. In another example, a transition zone location can be varied based on whether the catheter device is intended to femoral or radial access. Transition zones are located at an interface between the occlusion balloon and the lumen.

The catheter device 210 can be safely positioned in the anterior or posterior circulation. It can be safely used via a radial approach 250a, with balloon deflation commensurate with safe removal through the upper extremity without damaging corresponding arteries. The catheter device 210 can have sensors distal to the balloon to allow for inflation without using contrast agent. Instead, a practitioner can view the distal blood pressure waveform pulsatility change of the subject 250 on a display 230 as the balloon is inflated.

In one example, an intermediate catheter with embedded pressure sensors can be oriented distal and proximal to an occlusion balloon. The occlusion balloon can inflate sufficiently to provide protective occlusion and to effectively perform mechanical thrombectomy. The balloon can be inflated and deflated automatically, based on algorithms responding to arterial waveforms, allowing for dynamic adjustment of inflation to maintain optimal stasis of blood-flow as the catheter and balloon passes through arterial segments with variable diameter. As an example, a specific volume of saline can be delivered to both the distal and proximal balloon based on information gathered by the pressure sensors, one located downstream and one upstream of the clot site. This technology can enhance margins for clot retrieval, increase access at distal occlusion sites with better ease and safety, and minimize thrombus fragmentation. Although exact dimensions can vary, the distal pressure sensor or sensors can generally be placed within about 0 mm to 1 cm, and most often 0.2 mm to 1 cm, of an adjacent surface of the nearest occlusion balloon.

In another embodiment, as illustrated in FIG. 3a, a catheter device 300a can comprise a longitudinal lumen 304 having a proximal end 304a and a distal end 304b. In one optional aspect, the catheter device 300a can be inserted through an optional guide catheter 306. In one aspect, the distal end 304b can be capable of insertion into at least the internal carotid artery.

The catheter device 30a can have various properties to allow insertion into the narrower and more fragile cerebral vasculature. A catheter having excessive stiffness, although suitable with some contrast agents with a high viscosity, can prevent access to the cerebral vasculature. To allow access to the cerebral vasculature, the diameter of the lumen 304 can be reduced and the use of contrast agent can be avoided using one or more pressure sensors 310a, 310b to provide dynamic inflation and deflation of one or more occlusion balloons 320a, 320b. Notably, at least one pressure sensor 310a can be oriented on the guide wire 306 proximal and adjacent to the balloon 320a. Although exact dimensions may vary the pressure sensors can be within about 10 mm, and often within about 5 mm of a nearest surface of the occlusion balloon.

In one example, the catheter 300a can include a longitudinal lumen 304 of any suitable aspiration catheter. The catheter 300a can comprise a longitudinal lumen 304 used to extract a thrombus by connecting the longitudinal lumen 304 to a syringe or other extraction volume on negative pressure for aspiration. The catheter 300a can comprise additional concentric lumens such as a lumen for a guidewire, a microcatheter, a stent retriever, a suction source, and any other suitable tools.

In some cases, the catheter 300a can have a variable stiffness along a portion of a length of the catheter 300a. For example, the variable stiffness can include a catheter tip 304c stiffness that is less than a catheter shaft stiffness. In this manner, a proximal portion of the catheter can be stiffer than a distal portion so as to facilitate insertion along an entire vascular pathway to the target cerebral tissue.

The catheter device 300a can be a sterile single-use device comprising any suitable material. A suitable material for the catheter 300a can have various biological properties including but not limited to: biocompatibility (e.g., does not produce an inflammatory response when in contact with blood vessels), non-thrombogenicity (e.g., coating with a non-thrombogenic material such as heparin), non-mutagenic, non-toxic, biofilm resistant, microbial resistant, compliance, conformability, the like, or a combination thereof.

A suitable material for the catheter 300a can have various physical properties including but not limited to: a suitable tensile strength sufficient to withstand applied torque used in a inserting the catheter 300a to a thrombus location (torque control can be provided with a double-stranded steel braid), compression resistance to maintain a suitable shape (e.g., a high modulus and kink resistance), adequate flexibility to maneuver through blood vessels (a suitable modulus at the distal end to achieve a greater flexibility compared to the proximal end), a low coefficient of friction to allow movement of the catheter 300a through the vasculature, radiopaque properties, the like, or a combination thereof.

The variability of the stiffness of the catheter 300a can be achieved by bonding sections of different moduli together. In one example, the stiffness of the proximal end can range from about 100,000 psi to about 300,000 psi. In another example, the stiffness of the distal end can range from about 100 psi to about 5,000 psi. In another example, the stiffness of the distal end can range from about 100 psi to about 1,000 psi. In another example, the stiffness of the distal end can range from about 100 psi to about 500 psi.

In one more example, the catheter 300a can have various chemical properties, e.g., the catheter 300a can comprise a material that satisfies United States Pharmacopeia (USP) Class VI classifications. The catheter can be sterilized using various methods such as ethylene oxide (EtO), Sterrad, Steris System 1, Cidex OPA processes, the like, or a combination thereof. The material for the catheter 300a can accept a suitable coating to prevent microbial growth or thrombus-generation.

In one example, the material for the catheter 300a can comprise, but is not limited to, one or more of: polyurethanes (e.g., polyester-based polyurethanes, polyether-based polyurethanes, poly-carbonate-based polyurethanes having a suitable durometer, e.g., 75 Shore A to 75 Shore D, or thermoplastic polyurethanes, or the like), polyamides (e.g., nylon 11, nylon 12, or the like), fluoropolymers (e.g., polytetrafluorethylene (PTFE)), polyolefins, PVC, polyimides, polyetheretherketone (PEEK), the like, or a combination thereof. In one example, the catheter 300a can comprise a hydrophilic coating. Suitable hydrophilic coatings can include but are not limited to: polyethylene terephthalate (PET), PTFE, polyethylene (PE), the like, or a combination thereof.

In one aspect, the distal end 304b is capable of insertion into at least one of the anterior cerebral arteries, anterior communicating arteries, and the middle cerebral arteries. Further, the distal end can be capable of insertion into one or more of the internal carotid arteries, anterior cerebral arteries, middle cerebral arteries, vertebral arteries, basilar arteries, posterior cerebral arteries, and their branches. In one example, the catheter 300a can have a usable length for a procedure of from about 50 cm to about 150 cm. In one example, the usable length for a procedure can be from about 120 cm to about 145. In another example, the usable length for a procedure can be from about 130 cm to about 135 cm. In another example, the usable length for a procedure can be about 132 cm.

In one example, the inner and outer diameter of the catheter 300a can be selected to provide access to at least one of the anterior cerebral arteries, anterior communicating arteries, and the middle cerebral arteries. In one example, the inner diameter of the catheter can be about 1.37 mm (e.g., about 4 in French Gauge), and the outer diameter can be about 1.78 mm (e.g., about 5 in French Gauge). As a general guideline, the outer diameter of the catheter at the distal end can be from 0.9 mm to 2.2 mm, and most often 1 mm to 1.9 mm.

In one aspect, the catheter can comprise a soft radiopaque tip that facilitates vessel engagement and catheter placement into at least one of the anterior cerebral arteries, anterior communicating arteries, and the middle cerebral arteries. The smaller diameter of the catheter 300a and the variable stiffness with the softer tip 304c can allow the catheter 300a to be used for radial access. In one aspect, the catheter 300a can be adapted for radial access and can further comprise a radial pressure sensor oriented along a radial portion of the longitudinal lumen 304. In one aspect, a catheter 300a for use through the radial artery can use dynamic inflation and deflation of the occlusion balloon and by including inflation and deflation lumens that are reduced in size to allow for radial access. The radial pressure sensor can provide dynamic feedback including radial blood pressure data to a controller that can adjust the inflation or deflation of one or more occlusion balloons as the catheter is inserted throughout the radial vasculature.

In one aspect, the catheter device 300 can comprise an occlusion balloon 320b connected to the distal end 304b. In one example, the occlusion balloon 320b can be operable to occlude blood flow in a blood vessel 302 by inflation and deflation using a pressurization fluid.

In one example, the occlusion material can be formed of a material used for compliant balloons that can be inflated by volume not pressure. In one example, the one or more occlusion balloons 320a, 320b can comprise any suitable material for insertion into a cerebral artery including but not limited to: polyurethane, silicone, the like, or a combination thereof.

In one aspect, the one or more occlusion balloons 320a, 320b can be operable to inflate via injection of a selected volume of pressurization fluid into the occlusion balloon 320a, 320b or deflate via removal of at least a portion of the selected volume from the occlusion balloon 320a, 320b. In one example, the selected volume of pressurization fluid can be an amount of from about 1 μl to about 50 μl. In another example, the selected volume of pressurization fluid can be an amount of from about 1 μl to about 10 μl. In another example, the selected volume of pressurization fluid can be an amount of from about 1 μl to about 5 μl. In another aspect, the selected volume of pressurization fluid for insertion into the one or more occlusion balloons can be provided by the inflation control information received from a controller 350. In one aspect, an electronic syringe pump can be configured to provide the selected volume of pressurization fluid for insertion into the one or more occlusion balloons 320a, 320b. Due to the particularly fragile cerebral vasculature the dynamic volume control of the occlusion balloons can have a high precision and low volumetric variability. In one example, adjustments to occlusion volume can be controlled to within 0.05 μl to 5 μl and most often 1 μL to 3 μl.

In another example, the pressurization fluid can comprise a viscosity of from about 0.5 centipoise (cP) and 1.5 cP, and in some cases less than 1.4 cP, at STP. In one example, the pressurization fluid can have an osmolarity of from about 270 mOsm/L to about 330 mOsm/L. In another example, the saline can have a pH of from about 7.0 to about 7.5. In one example, the pressurization fluid can be saline. In one example, the saline can have a sodium chloride concentration of about 0.9% w/v.

In another aspect, the pressurization fluid can be a suitable liquid that is substantially free of contrast agent. A contrast agent can include any suitable agent that can be used to temporarily change the way x-rays, computed tomography (CT), magnetic resonance (MR) imaging, and ultrasound interact with the body. In one example, contrast agents can include but are not limited to: iodine-based and barium sulfate compounds used in x-ray and CT imaging exams; gadolinium; the like; or a combination thereof. Thus, the pressurization fluid can advantageously be a saline solution or CO₂, for example. In another aspect, the one or more occlusion balloons 320a, 320b can be configured to dynamically adjust balloon inflation or deflation to maintain occlusion during movement of the catheter 300a in the blood vessel 302 based on feedback from the one or more pressure sensors 310a, 310b. Dynamic inflation of the one or more balloons 320a, 320b can maintain sufficient occlusion without damaging the vessel walls 302a, 302b. For example, the diameter of the vasculature can change from a radial access position to an internal carotid artery to an anterior cerebral artery, or a middle cerebral artery, or a posterior cerebral artery. The diameter of the vasculature can also change within a specific cerebral artery. For example, the diameter of the middle cerebral artery can vary between different segments such as M1, M2, M3, and M4. The one or more occlusion balloons 320a, 320b can inflate in response to changing diametric vascular dimensions as the catheter is moved into or out of the cerebral arteries. In one aspect, dynamic adjustment of the occlusion balloons 320a, 320b can maintain sufficient occlusion without using a contrast agent to visualize occlusion. Using a catheter 300a without contrast agent allows for a reduction in the diameter of the catheter 300a and the stiffness of the distal end 304b to provide enhanced access into the cerebral vasculature (e.g., the anterior cerebral artery, or the middle cerebral artery, or the posterior cerebral artery).

In another example, the occlusion balloons 320a, 320b can be configured to have a degree of flexibility sufficient to be responsive to inflation and deflation adjustments in a selected time period without causing damage to tissue as the occlusion balloons 320a, 320b are moved through the cerebral vasculature. In one aspect, the occlusion balloon can be sufficiently responsive to inflation and deflation adjustments when the occlusion balloon can complete an inflation or deflation event within a selected time period, wherein the inflation or deflation event is the total time to complete inflation or deflation after receiving inflation control information from a controller. In one example, the selected time period can be less than one or more of: 10 ms, 5 ms, 1 ms, 100 μs, 50 μs, 10 μs, 1 μs, or a combination thereof.

In one aspect, the catheter device 300 can a pressure sensor 310b associated with the distal end 304b and operable to measure blood pressure data from at least one of a downstream and upstream location of the occlusion balloon 320b. In another aspect, the pressure sensor 304b can be operable to measure blood pressure data from at least one of a downstream 305b and upstream 305a location of the blood clot 305.

Although other pressure sensors can be suitable, the one or more pressure sensors 310a, 310b can most often be one of two different types: (i) an optical fiber based pressure sensor, or (ii) solid-state pressure sensors. In one example, the one or more pressure sensors 310a, 310b can be solid state pressure sensors. In one example, the one or more pressure sensors 310a, 310b can be an IntraSense® SMI-1B-48-150-BAUU pressure sensor having a pressure range of from about 460 mmHg to about 1260 mmHg, a sensor size of about 1 French, and pressure sensitivity of about 5 uV/V/mmHg. In another example, the one or more pressure sensors 310a, 310b can be a obtained from a different commercial vendors (e.g., Millar®).

In one aspect, the one or more pressure sensors 310a, 310b can be operable to measure blood pressure data within a margin of error of less than at least one of 2 mmHg, 1 mmHg, 0.5 mmHg, the like, or a combination thereof. In another aspect, the one or more pressure sensors 310a, 310b can be operable to provide blood pressure data, blood flow information, or blood pressure waveform pulsatility to a controller via microwires embedded in the catheter 300a. In another aspect, the one or more pressure sensors 310a, 310b can be operable to measure blood pressure waveform pulsatility. In one example, the “waveform pulsatility” can be based on the pulsatility index (PI) as calculated the difference between the peak systolic velocity and the end diastolic volume divided by the mean flow velocity during the cardiac cycle. In one example, the PI of a cerebral artery (e.g., MCA) can be measured using Trans Cranial Doppler (TCD). As used herein, “blood flow information” can be blood perfusion (e.g., the volume of blood flowing through a certain mass (or volume) or tissue per unit time, with units of mL/(mL*min). As used herein, blood pressure can include the systolic blood pressure, the diastolic blood pressure, the like, or a combination thereof.

In another aspect, one or more of blood pressure data, blood flow information, or blood pressure waveform pulsatility can be transmitted to a controller 350 via a wireless or wired connection 355. In one example, the controller 350 can be operable to receive one or more of blood pressure data, blood flow information, or blood pressure waveform pulsatility from the one or more pressure sensors 310a, 310b. In one aspect, the controller 350 can be operable to generate inflation control information based on one or more of blood pressure data, blood flow information, or blood pressure waveform pulsatility. In one aspect, the controller can be operable to transmit the inflation control information to the occlusion balloon 320b. As used herein, “inflation control information” refers to a control signal from the controller that regulates the inflation or deflation of the one or more occlusion balloons 320a, 320b. In one aspect, the one or more occlusion balloons 320a, 320b can be operable to inflate or deflate based on the inflation control information by changing a selected volume of the pressurization fluid within the one or more occlusion balloons 320a, 320b. In another aspect, the inflation control information can be at the controller from one or more of the blood pressure data, the blood flow information, or blood pressure waveform pulsatility using a pressure-tracing algorithm. As one specific example, development of a tardus parvus waveform during the procedure can indicate successful occlusion. This can involve a prolonged systolic upslope, a rounded peak, and a prolonged return to diastolic pressure.

In another example, the blood clot can be captured via a stent retriever or via aspiration. In one embodiment, as illustrated in FIG. 3b, the catheter device 300b can further comprise a stent retriever 330 oriented within the lumen and deployable to capture and remove a blood clot 305. In another embodiment, as illustrated in FIG. 3c, the catheter device 300c can further comprise a suction source 370 fluidly connected 375 to the lumen 304 and configured to remove debris from a blood clot 305 via aspiration.

In one embodiment, as illustrated in FIG. 3d, the catheter device 300d can comprise a proximal occlusion balloon 320a oriented adjacent the distal end 304b. In some cases, as illustrated in FIG. 3a, the catheter device 300a can comprise both a distal occlusion balloon 320b oriented at the distal end 304b and a proximal occlusion balloon 320a oriented adjacent the distal end 304b. In one aspect, the distal occlusion balloon 304b can be operable to prevent downstream 305b passage of fragmented blood clots that break off from the blood clot 305.

In another embodiment, as illustrated in FIG. 3e, the catheter device 300e can further comprise an outer catheter 380. In this example, the outer catheter can be a guide catheter that can have a diameter that is operable for access outside of the cerebral arteries and the inner catheter can be configured for access to the cerebral arteries as discussed herein.

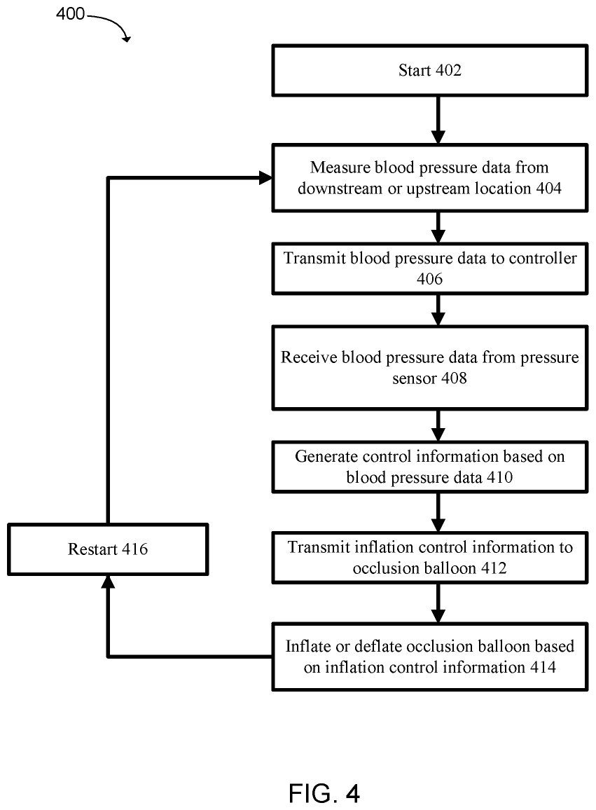

In one embodiment, as depicted in FIG. 4, functionality 400 for adjustment of balloon inflation or deflation to maintain balloon occlusion during movement of the catheter in the blood vessel can include a start operation 402 or an activation operation. Another operation can include measuring blood pressure data, at one or more pressure sensors, from a downstream or upstream location of a blood clot or of an occlusion balloon, as shown in in operation 404. Another operation can include transmitting, from the pressure sensor, the blood pressure data to a controller, as shown in operation 406. Another operation can include receiving, at a controller, blood pressure data from the pressure sensor, as shown in operation 408. Another operation can include generating control information at the controller based on blood pressure data received from the one or more pressure sensors, as shown in operation 410. Another operation can include transmitting the control information from the controller to a suitable flow control device for delivering or withdrawing pressurization fluid from the occlusion balloon, as shown in operation 412. Another operation can include inflating or deflating the occlusion balloon based on the inflation control information received from the controller, as shown in operation 414. After the occlusion balloon has been inflated or deflated, the restart operation 416 can loop back to operation 404. In one example, the blood pressure data and the blood flow information collected from the one or more pressure sensors can be displayed on a graphical user interface that can be electrically connected to the controller.

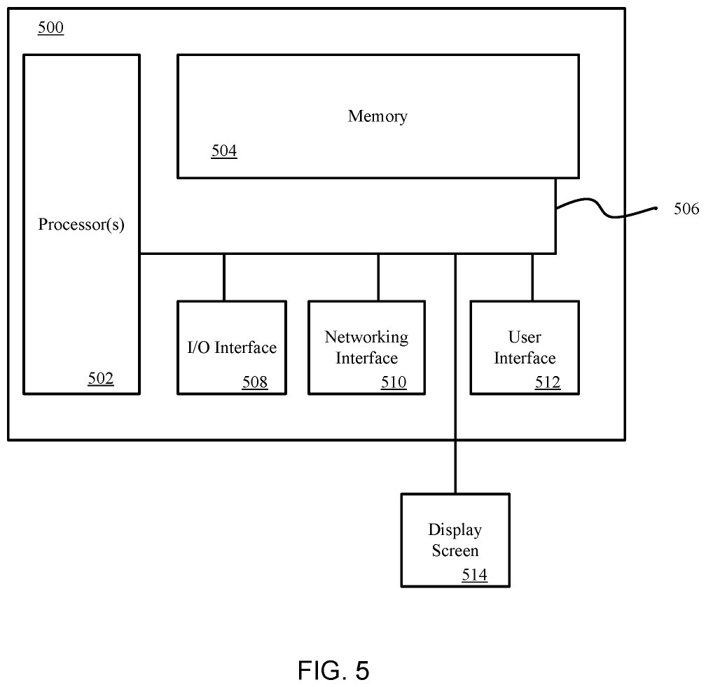

FIG. 5 illustrates a general computing system or device 500 that can be employed in the present technology. The computing system 500 can include a processor 502 in communication with a memory 504. The memory 504 can include any device, combination of devices, circuitry, and the like that is capable of storing, accessing, organizing, and/or retrieving data. Non-limiting examples include SANs (Storage Area Network), cloud storage networks, volatile or non-volatile RAM, phase change memory, optical media, hard-drive type media, and the like, including combinations thereof.

The computing system or device 500 additionally includes a local communication interface 518 for connectivity 506 between the various components of the system. For example, the local communication interface can be a local data bus and/or any related address or control busses as may be desired.

The computing system or device 500 can also include an I/O (input/output) interface 508 for controlling the I/O functions of the system, as well as for I/O connectivity to devices outside of the computing system 500. A network interface 516 can also be included for network connectivity. The network interface 510 can control network communications both within the system and outside of the system. The network interface can include a wired interface, a wireless interface, a Bluetooth interface, optical interface, and the like, including appropriate combinations thereof. Furthermore, the computing system 500 can additionally include a user interface 512, a display device 540, as well as various other components that would be beneficial for such a system.

The processor 502 can be a single or multiple processors, and the memory 504 can be a single or multiple memories. The local communication interface 518 can be used as a pathway to facilitate communication between any of a single processor, multiple processors, a single memory, multiple memories, the various interfaces, and the like, in any useful combination.

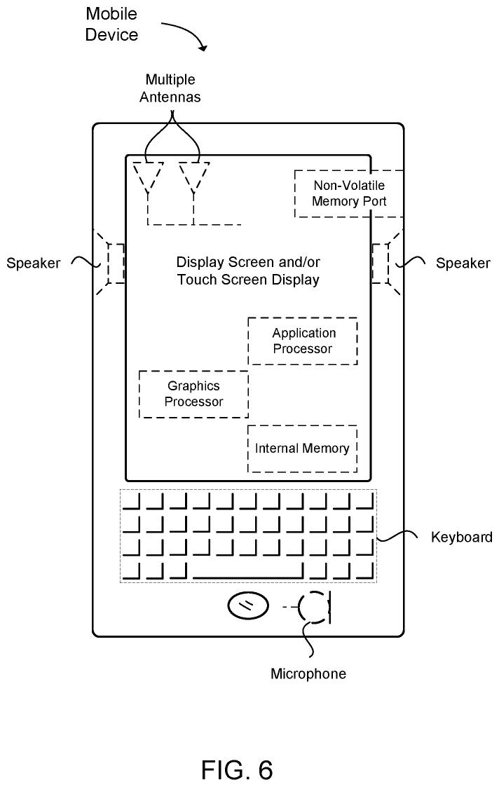

FIG. 6 provides an example illustration of the wireless device, such as a user equipment (UE), a mobile station (MS), a mobile wireless device, a mobile communication device, a tablet, a handset, or other type of wireless device. The wireless device can include one or more antennas configured to communicate with a node, macro node, low power node (LPN), or, transmission station, such as a base station (BS), an evolved Node B (eNB), a baseband processing unit (BBU), a remote radio head (RRH), a remote radio equipment (RRE), a relay station (RS), a radio equipment (RE), or other type of wireless wide area network (WWAN) access point. The wireless device can be configured to communicate using at least one wireless communication standard such as, but not limited to, 3GPP LTE, WiMAX, High Speed Packet Access (HSPA), Bluetooth, and WiFi. The wireless device can communicate using separate antennas for each wireless communication standard or shared antennas for multiple wireless communication standards. The wireless device can communicate in a wireless local area network (WLAN), a wireless personal area network (WPAN), and/or a WWAN. The wireless device can also comprise a wireless modem. The wireless modem can comprise, for example, a wireless radio transceiver and baseband circuitry (e.g., a baseband processor). The wireless modem can, in one example, modulate signals that the wireless device transmits via the one or more antennas and demodulate signals that the wireless device receives via the one or more antennas.

FIG. 6 also provides an illustration of a microphone and one or more speakers that can be used for audio input and output from the wireless device. The display screen can be a liquid crystal display (LCD) screen, or other type of display screen such as an organic light emitting diode (OLED) display. The display screen can be configured as a touch screen. The touch screen can use capacitive, resistive, or another type of touch screen technology. An application processor and a graphics processor can be coupled to internal memory to provide processing and display capabilities. A non-volatile memory port can also be used to provide data input/output options to a user. The non-volatile memory port can also be used to expand the memory capabilities of the wireless device. A keyboard can be integrated with the wireless device or wirelessly connected to the wireless device to provide additional user input. A virtual keyboard can also be provided using the touch screen.

Various techniques, or certain aspects or portions thereof, can take the form of program code (i.e., instructions) embodied in tangible media, such as floppy diskettes, compact disc-read-only memory (CD-ROMs), hard drives, non-transitory computer readable storage medium, or any other machine-readable storage medium wherein, when the program code is loaded into and executed by a machine, such as a computer, the machine becomes an apparatus for practicing the various techniques. Circuitry can include hardware, firmware, program code, executable code, computer instructions, and/or software. A non-transitory computer readable storage medium can be a computer readable storage medium that does not include signal. In the case of program code execution on programmable computers, the computing device can include a processor, a storage medium readable by the processor (including volatile and non-volatile memory and/or storage elements), at least one input device, and at least one output device. The volatile and non-volatile memory and/or storage elements can be a random-access memory (RAM), erasable programmable read only memory (EPROM), flash drive, optical drive, magnetic hard drive, solid state drive, or other medium for storing electronic data. The low energy fixed location node, wireless device, and location server can also include a transceiver module (i.e., transceiver), a counter module (i.e., counter), a processing module (i.e., processor), and/or a clock module (i.e., clock) or timer module (i.e., timer). One or more programs that can implement or utilize the various techniques described herein can use an application programming interface (API), reusable controls, and the like. Such programs can be implemented in a high-level procedural or object-oriented programming language to communicate with a computer system. However, the program(s) can be implemented in assembly or machine language, if desired. In any case, the language can be a compiled or interpreted language, and combined with hardware implementations.

As used herein, the term processor can include general purpose processors, specialized processors such as VLSI, FPGAs, or other types of specialized processors, as well as base band processors used in transceivers to send, receive, and process wireless communications.

It should be understood that many of the functional units described in this specification have been labeled as modules, in order to more particularly emphasize their implementation independence. For example, a module can be implemented as a hardware circuit comprising custom very-large-scale integration (VLSI) circuits or gate arrays, off-the-shelf semiconductors such as logic chips, transistors, or other discrete components. A module can also be implemented in programmable hardware devices such as field programmable gate arrays, programmable array logic, programmable logic devices or the like.

In one example, multiple hardware circuits or multiple processors can be used to implement the functional units described in this specification. For example, a first hardware circuit or a first processor can be used to perform processing operations and a second hardware circuit or a second processor (e.g., a transceiver or a baseband processor) can be used to communicate with other entities. The first hardware circuit and the second hardware circuit can be incorporated into a single hardware circuit, or alternatively, the first hardware circuit and the second hardware circuit can be separate hardware circuits.

Modules can also be implemented in software for execution by various types of processors. An identified module of executable code can, for instance, comprise one or more physical or logical blocks of computer instructions, which can, for instance, be organized as an object, procedure, or function. Nevertheless, the executables of an identified module need not be physically located together but can comprise disparate instructions stored in different locations which, when joined logically together, comprise the module and achieve the stated purpose for the module.

Indeed, a module of executable code can be a single instruction, or many instructions, and can even be distributed over several different code segments, among different programs, and across several memory devices. Similarly, operational data can be identified and illustrated herein within modules and can be embodied in any suitable form and organized within any suitable type of data structure. The operational data can be collected as a single data set or can be distributed over different locations including over different storage devices, and can exist, at least partially, merely as electronic signals on a system or network. The modules can be passive or active, including agents operable to perform desired functions.

### Methods of Using the Catheter Device

Disclosed herein is a neurovascular mechanical thrombectomy device that can be used in the treatment of acute ischemic stroke. The device can be delivered into the neuro-vasculature with an endovascular approach. The thrombus can be mechanically removed to restore blood flow in the neuro-vasculature.

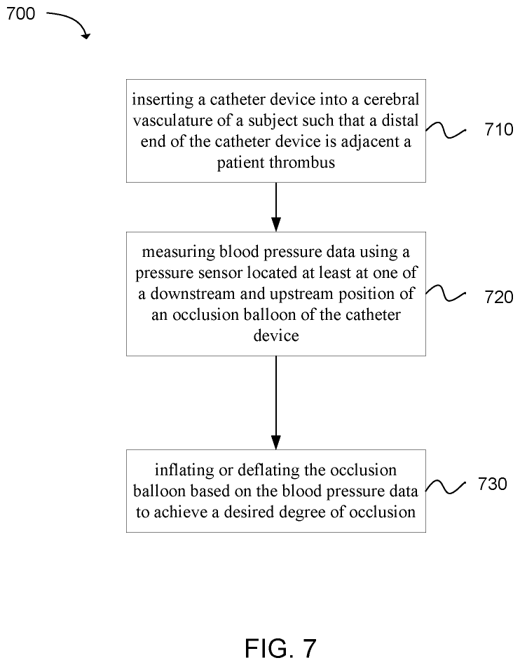

In one embodiment, as illustrated in FIG. 7, a method 700 of using a catheter device to treat cerebral thrombectomy in a subject can comprise inserting a catheter device into a cerebral vasculature of a subject such that a distal end of the catheter device is adjacent a patient thrombus, as shown in block 710. The method can further comprise measuring blood pressure data using a pressure sensor located at least at one of a downstream and upstream position of an occlusion balloon of the catheter device, as shown in block 720. The method can further comprise inflating or deflating the occlusion balloon based on the blood pressure data to achieve a desired degree of occlusion, as shown in block 730.

In another aspect, the method can comprise dynamically adjusting a balloon inflation or deflation to maintain occlusion during movement of the catheter in the cerebral vasculature based on feedback from the pressure sensor. In another aspect, the method can comprise providing the catheter with a variable stiffness to allow insertion into the cerebral vasculature of the subject. In another aspect, the method can comprise providing the catheter with a size allowing radial access to the subject. In another aspect, the method can comprise removing the thrombus from the subject. In another aspect, the method can comprise generating inflation control information from the blood pressure data and pressure-tracing algorithms to adjust the inflation or deflation of the occlusion balloon. In another aspect, the method can comprise measuring blood pressure waveform pulsatility and blood flow information using the pressure sensor.

### EXAMPLES

The following examples are provided to promote a more clear understanding of certain embodiments of the present disclosure, and are in no way meant as a limitation thereon.

### Example 1: Generated Pressures of a Catheter Device

A catheter device disclosed herein can have a balloon-rated burst pressure of >600 PSI. For a provided flow rate, the generated pressures are provided in Table 1.

TABLE 1
Generated Pressures at Given Flow Rate

| | | | | | |
|---|---|---|---|---|---|
| mL/s | 4 | 6 | 8 | 10 | 15 |
| psi | 111 | 190 | 278 | 369 | 599 |
| Kg/cm² | 8 | 13 | 20 | 26 | 42 |

### Example 2: Use of a Catheter Device for Treatment of Acute Ischemic Stroke

The radial artery is punctured with a sharp hollow needle. A guidewire is advanced through the lumen of the needle and the needle is withdrawn. A sheath is passed into the blood vessel using the guidewire. An outer catheter 380 is used to introduce the catheter device 300e into the radial artery. The catheter device 300e can be guided to the cerebral artery to a location of a thrombus 305 without the use of a contrast agent. The pressures sensors 310a and 310b provide real-time blood pressure data to a controller 350.

The controller generates inflation control information and signals the occlusion balloon 320a to inflate when the degree of occlusion resulting from occlusion balloon 320a or 320b is below a selected level of occlusion and to deflate when the degree of occlusion resulting from occlusion balloon 320a or 320b is above a selected level of occlusion that may cause injury to the vessel walls 302a, 302b.

Once the catheter device is adjacent the thrombus 305, a suction source can be used to aspirate the thrombus. The thrombus 305 can be prevented from fragmentation and downstream passage via occlusion balloon 320b. The degree of downstream blood flow can be reduced using occlusion balloon 320a. A stent retriever 330 can also be used to retrieve the thrombus from the cerebral artery.

After the procedure has been completed, the catheter device can be removed from the cerebral artery through the radial access by suitable inflation and deflation of the occlusion balloons 320a, 320b based on inflation control information from the controller 350.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Reference was made to the examples illustrated in the drawings and specific language was used herein to describe the same. It will nevertheless be understood that no limitation of the scope of the technology is thereby intended. Alterations and further modifications of the features illustrated herein, and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the disclosure with reference to specific exemplary embodiments. However, it will be appreciated that various modifications and changes can be made without departing from the scope of the present disclosure as set forth in the appended claims. The detailed description and accompanying drawings are to be regarded as merely illustrative, rather than as restrictive, and all such modifications or changes, if any, are intended to fall within the scope of the present disclosure as described and set forth herein.

What is claimed is:

1. A catheter device comprising:
a catheter including a longitudinal lumen and having a proximal end and a distal end, wherein the distal end is capable of insertion into at least the internal carotid artery;
an occlusion balloon connected to the distal end and operable to occlude blood flow in a blood vessel by inflation and deflation using a pressurization fluid;
a pressure sensor associated with the distal end and operable to measure blood pressure data from at least one of a downstream and upstream location of the occlusion balloon and transmit the blood pressure data to a controller; and
the controller operable to:
receive the blood pressure data from the pressure sensor;
generate inflation control information based on the blood pressure data; and
transmit the inflation control information to the occlusion balloon,
wherein the occlusion balloon is operable to inflate or deflate based on the inflation control information by changing a selected volume of the pressurization fluid within the occlusion balloon.

2. The catheter device of claim 1, wherein the occlusion balloon is operable to inflate via injection of the selected volume of pressurization fluid into the occlusion balloon or deflate via removal of at least a portion of the selected volume from the occlusion balloon.

3. The catheter device of claim 2, wherein the inflation control information includes a value for the selected volume.

4. The catheter device of claim 2, wherein the pressurization fluid is saline and free of contrast agent.

5. The catheter device of claim 1, wherein the occlusion balloon is configured to dynamically adjust balloon inflation or deflation to maintain occlusion during movement of the catheter in the blood vessel based on feedback from the pressure sensor.

6. The catheter device of claim 1, wherein the occlusion balloon is operable to maintain sufficient occlusion without using a contrast agent to visualize occlusion.

7. The catheter device of claim 1, wherein the catheter has a variable stiffness along a portion of a length of the catheter, wherein the variable stiffness comprises a catheter tip stiffness that is less than a catheter shaft stiffness.

8. The catheter device of claim 1, wherein the distal end is capable of insertion into at least one of the anterior cerebral arteries, anterior communicating arteries, and the middle cerebral arteries.

9. The catheter device of claim 1, wherein the catheter is adapted for radial access and further comprises a radial pressure sensor oriented along a radial portion of the longitudinal lumen, wherein the radial portion is in the radial artery.

10. The catheter device of claim 1, wherein the pressure sensor is operable to measure blood pressure data within a margin of error of less than 1 mmHg.

11. The catheter device of claim 1, wherein the pressure sensor is operable to measure blood pressure waveform pulsatility and blood flow information.

12. The catheter device of claim 1, wherein the inflation control information is generated from the blood pressure data and pressure-tracing algorithms.

13. The catheter device of claim 1, further comprising a hydrophilic coating including polyethylene terephthalate (PET), polytetrafluorethylene (PTFE), polyethylene (PE), or a combination thereof.

14. The catheter device of claim 1, wherein the occlusion balloon is a proximal occlusion balloon oriented adjacent the distal end, and the catheter device further comprises a distal occlusion balloon oriented at the distal end and operable to prevent downstream passage of fragmented blood clots.

15. The catheter device of claim 1, further comprising a stent retriever oriented within the lumen and deployable to capture and remove a blood clot.

16. The catheter device of claim 1, further comprising a suction source fluidly connected to the lumen and configured to remove debris from a blood clot via aspiration.

17. The catheter device of claim 1, further comprising a graphical user interface electrically connected to the controller and operable to display blood pressure data and blood flow information.

18. The catheter device of claim 1, wherein the catheter is operable for embolization delivery.

19. A method of using a catheter device to treat cerebral thrombectomy in a subject comprising:
inserting a catheter device into a cerebral vasculature of a subject such that a distal end of the catheter device is adjacent a patient thrombus;
measuring blood pressure data using a pressure sensor located at least at one of a downstream and upstream position of an occlusion balloon of the catheter device; and
inflating or deflating the occlusion balloon based on the blood pressure data to achieve a desired degree of occlusion.

20. The method of claim 19, further comprising:
dynamically adjusting a balloon inflation or deflation to maintain occlusion during movement of the catheter in the cerebral vasculature based on feedback from the pressure sensor.

21. The method of claim 19, further comprising:
providing the catheter with a variable stiffness to allow insertion into the cerebral vasculature of the subject.

22. The method of claim 19, further comprising:
providing the catheter with a size allowing radial access to the subject.

23. The method of claim 19, further comprising removing the thrombus.

24. The method of claim 19, further comprising:
generating inflation control information from the blood pressure data and pressure-tracing algorithms to adjust the inflation or deflation of the occlusion balloon.

25. The method of claim 19, further comprising:
measure blood pressure waveform pulsatility and blood flow information using the pressure sensor.
